(19) United States
(12) Patent Application Publication
(10) Pub. No.: US 2009/0104141 A1
(43) Pub. Date: Apr. 23, 2009

(54) ISOTHERMAL PREPARATION OF HEAT-RESISTANT GELLAN GELS WITH REDUCED SYNERESIS

(75) Inventors: Shinya Ikeda, San Diego, CA (US); Ronnie Yuan, San Diego, CA (US); Todd Anthony Talashek, San DIego, CA (US)

Correspondence Address: CP Kelco US, INC, 1000 PARKWOOD CIRCLE, SUITE 1000, ATLANTA, GA, 30339, US

(73) Assignee: CP KELCO US, INC., Atlanta, GA (US)

(21) Appl. No.: 11/875,677

(22) Filed: Oct. 19, 2007

(51) Int. Cl. A61L 9/04; B01J 13/00; C08L 5/08

(52) U.S. Cl. 424/76.4; 512/4; 516/102; 516/106; 516/107

(57) ABSTRACT

A gel comprising a deacylated gellan gum, an effective amount of a sequestrant, an effective amount of a syneresis control agent, and a gelation inducer is described. Also methods for making the gel are disclosed including a method for forming heat-resistant gels comprising mixing deacylated gellan gum and xyloglucan; hydrating the blend; resting the hydrated blend until a gel forms. The gels can be used in a variety of applications such as air freshener gels.

### BACKGROUND

Polysaccharides, which are also referred to as gums, are primarily used to thicken or gel aqueous solutions. Polysaccharides that are produced by microorganisms of the genus Sphingomonas are also referred to as sphingans. Gums are frequently classified into two groups: thickeners and gelling agents. Typical thickeners include starches, guar gum, carboxymethylcellulose, alginate, methylcellulose, xanthan gum, gum karaya, and gum tragacanth. Common gelling agents include gellan gum, gelatin, starch, alginate, pectin, carrageenan, agar, and methylcellulose.

Gelling agents are used in the food industry in a variety of applications, including confectionary jellies, jams, dessert gels, icings, dairy products, beverages, and the like. Additionally, gelling agents can be used as components of microbiological media. Gelling agents differ in the conditions under which they may be used and in the texture of the gels they form. These distinctive properties of gels have led to the widespread use of certain gelling agents in particular products (e.g., starch in confectionary jellies; gelatin in dessert gels; agar in icings; and alginate in pimento strips).

One particularly useful gelling agent is gellan gum, which is a capsular polysaccharide produced by the bacterium Sphingomonas elodea, ATCC 31461, and strains derived from this species. The constituent sugars of gellan gum are glucose, glucuronic acid, and rhamnose in the molar ratio of 2:1:1. These are linked together to give a primary structure comprising a linear tetrasaccharide repeat unit (O'Neill M. A., et al., “Structure of the acidic extracellular gelling polysaccharide produced by Pseudomonas elodea,” Carbohydrate Res., 124(1):123-133 (1983); Jansson, P. E., et al., “Structural studies of gellan gum, an extracellular polysaccharide elaborated by Pseudomonas elodea,” Carbohydrate Res., 124(1):135-139 (1983)). In the native or high acyl (“HA”) form, two acyl substituents, acetate and glycerate, are present. Both substituents are located on the same glucose residue and, on average, there is one glycerate per repeat unit and one acetate per every two repeat units. In the low acyl (“LA”) form, most of the acyl groups have been removed to produce a linear repeat unit substantially lacking such groups. X-ray diffraction analysis shows that gellan gum exists as a three-fold, left-handed, parallel double helix (Chandraskaran, R., et al., “The crystal structure of gellan,” Carbohydrate Res., 175(11):1-15 (1988); Chandraskaran, R., et al., “Cation interactions in gellan: An x-ray study of the potassium salt,” Carbohydrate Res., 181:23-40 (1988)).

LA gellan gums form gels when cooled in the presence of gel-promoting cations, preferably divalent cations, such as calcium and magnesium. The gels formed are firm and brittle. HA gellan gums do not require the presence of cations for gel formation, and the gels formed have structural and rheological characteristics which are significantly affected by the acyl substituents. Thus, the properties of HA gellan gums differ significantly from those of LA gellan gums. HA gellan gum gels are typically soft and flexible and lack thermal hysteresis.

Gellan gum displays different characteristics depending upon the method of recovery from the fermentation broth from which they are made. Direct recovery from the fermentation broth yields gellan in its native or high-acyl form. Isolation of gellan in this native or high-acyl form yields a soft, flexible, elastic gel. Gellan may be deacylated to provide gellan in its low acyl form. Isolation of gellan in this low acyl form yields a hard, firm, brittle gel. Blends of native and low acyl gellan produce gels of intermediate texture.

Currently, gels, such as air freshener gels, are formed by mixing an aqueous solution containing a gelling agent (such as gellan gum or carrageenan), gel promoting ions, and a fragrance. The solution is heated and cooled to form a gel. A disadvantage of this method is the heating step can drive off or degrade the fragrance. The volatile fragrance is the most expensive ingredient in most air freshener gels. Thus, alternative compositions and/or processes which can decrease or avoid this disadvantage are desirable.

### SUMMARY OF THE INVENTION

Described herein is the use of deacylated gellan gum for isothermally preparing heat-resistant gels with little or no syneresis without involving any heat treatments.

Described herein is a gel comprising a deacylated gellan gum, an effective amount of a sequestrant, an effective amount of a syneresis control agent, and a gelation inducer comprising an acidifier. The syneresis control agent can be a xyloglucan such as tamarind seed xyloglucan (TSX). The acidifier can be glucono-δ-lactone (GDL). The gel can further comprise water. The gel can also further comprise additional ingredients, such as fragrance.

Described herein is a process for preparing a gel comprising blending deacylated gellan with xyloglucan to form a blend; hydrating the blend to form a solution; resting the solution until a sufficient network structure is formed to maintain the shape of a gel. The xyloglucan can be TSX. The solution can further comprise a sequestrant. The solution can further comprise a gelation inducer comprising an acidifier. The process can be carried out without heat treatment.

Also described is a method of preparing a heat-resistant gel comprising blending a deacylated gellan gum, an effective amount of a sequestrant, an effective amount of a syneresis control agent, and a gelation inducer comprising an acidifier to form a blend; hydrating the blend with an amount of water; and resting the hydrated blend until a gel forms wherein the blending, hydrating and resting are performed essentially isothermally at ambient conditions. The syneresis control agent can be a xyloglucan such as tamarind seed xyloglucan (TSX). The acidifier can be glucono-δ-lactone (GDL). The gel can also further comprise additional ingredients, such as fragrance. Alternatively, the deacylated gellan gum, sequestrant, and syneresis control agent can be hydrated in any order with the gelation inducer comprising an acidifier being added last since this addition will begin gelation. Preferably, the hydrated blend is essentially homogeneous before the solution is allowed to rest until gel formation completes.

The invention includes a process for preparing a gel comprising the steps of isothermally hydrating deacylated gellan at ambient temperature; mixing the hydrated deacylated gellan gum and a tamarind seed xyloglucan; and gelling the mixture by adding a gelation inducer comprising an acidifier. The deacylated gellan can be hydrated at ambient temperatures in the presence of an effective amount of a sequestrant.

In a further aspect, the invention further includes compositions or products comprising a novel gel described herein. In addition, the invention includes a composition or product comprising a gel prepared by a method described herein.

For example, an air freshener gel can comprise a gel of the invention. An air freshener gel can further comprise fragrance.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 shows acidification profiles of 0.5% (top dashed line) and 1% (bottom solid line) GDL solutions containing 0.02% sodium citrate and 1.5% TSX from Example 1.

FIG. 2 shows a graph of temperature dependence of the storage modulus and loss modulus of an acid-set gel containing 0.5% KELCOGEL® gellan, 0.5% TSX, and 0.5% GDL from Example 1. Circles and triangles represent the storage and loss modulus, respectively. Open and solid symbols represent cooling and heating, respectively. Measurements were made two days after preparation to equilibrate pH.

FIG. 3 shows release of calcium ions in solutions containing 0.05% sodium citrate, 1.5% TSX, and 0.2% (top), 0.15% (middle), or 0.05% (bottom) calcium lactate from Example 2. Broken lines indicate stoichiometric values.

FIG. 4 shows release of calcium ions in solutions containing 0.5% GDL and 0.05% (top solid) or 0.03% (bottom dashed) CaHPO₄ from Example 2. Thin lines indicate pH values.

FIG. 5 shows a graph of temperature dependence of the storage modulus and loss modulus of 0.2% KELCOGEL® gellan mixed with 1.3% TSX (circle), 1.5% KELCOGEL® gellan alone (triangle), and 1.5% TSX alone (square) from Example 3. Open and solid symbols represent cooling and heating, respectively. The top two data sets are storage modulus values. The bottom data set is the loss modulus.

FIG. 6 shows a graph of the effects of mixing ratio on gel set temperature (open symbol) and melt temperature (closed symbol) (total gum level was 1.5%) from Example 3.

FIG. 7 shows a graph of the effects of mixing ratio on the storage modulus determined at 10° C. and loss modulus determined at 40° C. on initial cooling (total gum level was 1.5%) from Example 3.

### DETAILED DESCRIPTION OF THE INVENTION

Before the present compositions, articles, and/or methods are disclosed and described, it is to be understood that the aspects described below are not limited to specific embodiments; specific embodiments as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

It must be noted that, as used in the specification and the appended claims, the singular forms “a,” “an,” and “the” include plural referents unless the context clearly dictates otherwise. Thus, for example, reference to “an aqueous solution” includes mixtures of aqueous solutions; reference to “a gellan gum” includes mixtures of two or more such gums, and the like.

“Optional” or “optionally” means that the subsequently described event or circumstance may or may not occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### Compositions

A composition of the invention includes a heat-resistant acid-set gel with little or no syneresis, e.g., about equal to or less than 5%, 3%, 2%, 1%, or 0.5% syneresis. Described herein is a composition comprising a deacylated gellan gum, a sequestrant, a syneresis control agent (e.g., xyloglucan), and a gelation inducer comprising an acidifier. A composition of the invention can further comprise fragrance.

Deacylated gellan/xyloglucan blend systems that can be isothermally hydrated and gelled, e.g., at room temperature, are disclosed herein.

A composition of the invention comprises deacylated gellan gum. Deacylated gellan gum (e.g., KELCOGEL® gellan, CP Kelco, Atlanta, Ga.) is known in the art and is commercially available or can be produced by known methods. Any essentially fully deacylated gellan gum can be used. The amount of deacylated gellan in the gel is that which is effective to create a gel of the desired characteristics in the final product. For example, about 0.2 to about 0.75 wt % deacylated gellan gum can be used, preferably about 0.4 to about 0.5 wt %. A composition can comprise about 0.2, 0.3, 0.4, 0.45, 0.5, 0.6, or 0.7 wt % deacylated gellan gum. A gel comprising about >1% deacylated gellan gum may be too stiff or hard to be preferably used in an application such as an air freshener gel. One of ordinary skill in the art can choose a deacylated gellan gum and determine an amount thereof effective for a desired texture, viscosity, gel strength, etc.

A composition of the invention comprises a sequestrant. Sequestrants/chelating agents, e.g., sodium citrate, ethylenediaminetetraacetic acid (EDTA), sodium hexametaphosphate, are known in the art and are commercially available. One sequestrant or a mixture of sequestrants can be used. The amount of sequestrant used in a gel composition of the invention is an amount effective to chelate multivalent ions in the composition, particularly those in the water, to allow essentially complete ambient hydration of the deacylated gellan gum. For example, about 0.02 to about 0.05 wt % sequestrant can be used, preferably about 0.02 wt %. One of ordinary skill in the art can choose an appropriate sequestrant and determine an effective amount such that the deacylated gellan gum will hydrate at ambient conditions. For example, if the ion content of an aqueous solution used for hydration is determined, the amount of sequestrant can be calculated. It is believed an excess amount of sequestrant can be used without detrimentally affecting the composition, but it would not be cost-effective to add more sequestrant than necessary for the gellan to hydrate completely.

A composition of the invention comprises a syneresis control agent, e.g., xyloglucan, preferably tamarind seed xyloglucan (TSX). Preferably, the syneresis control agent also contributes to homogeneity of gelation.

Tamarind seed xyloglucan is a mucoadhesive polymer extracted from tamarind seeds (aka tamarind seed polysaccharide [TSP]) and has been described as a viscosity enhancer showing mucomimetic, mucoadhesive, and bioadhesive activities (e.g., M. F. Saettone, S. Burgalassi, E. Boldrini, P. Bianchini, and G. Luciani, 1997, international patent application PCT/IT97/00026). Xyloglucan extracted from tamarind seed is a polysaccharide which has a 1,4-β-D-glucan backbone partially substituted by 1,6-α-D-xylopyranosyl side chains, some of which are further substituted by 1,2-β-D-galactopyranosyl residue. TSX does not form a gel by itself unless a large amount of sugar and/or alcohol are added. Tamarind seed xyloglucan (TSX) has been found to promote gelation of deacylated gellan in the absence of additional ions.

It is believed xyloglucans other than TSX can be used in a composition of the invention. A syneresis control agent which provides effective control of water and does not interfere with the structure of the final product/negatively influence gelation can be used. One of ordinary skill in the art can determine an appropriate syneresis control agent to use in a composition of the invention.

TSX (or other xyloglucan) is commercially available. The amount of TSX (or other syneresis control agent) used in the gel composition is that which is effective to control the level of syneresis in the final product. For example, about 0.5 to about 1.5 wt % TSX can be used, preferably about 1.0 to about 1.5 wt %. A composition can comprise about 0.6, 0.7, 0.8, 0.9, 1.0, 1.2, 1.3, or 1.35 wt % TSX. A consumer generally desires a final product such as an air freshener gel to have no visible water exuding from the gel. Some syneresis would be acceptable to a final consumer to the extent the final product's aesthetics and functionality are not noticeably affected. One of ordinary skill in the art can determine an effective amount of syneresis control agent.

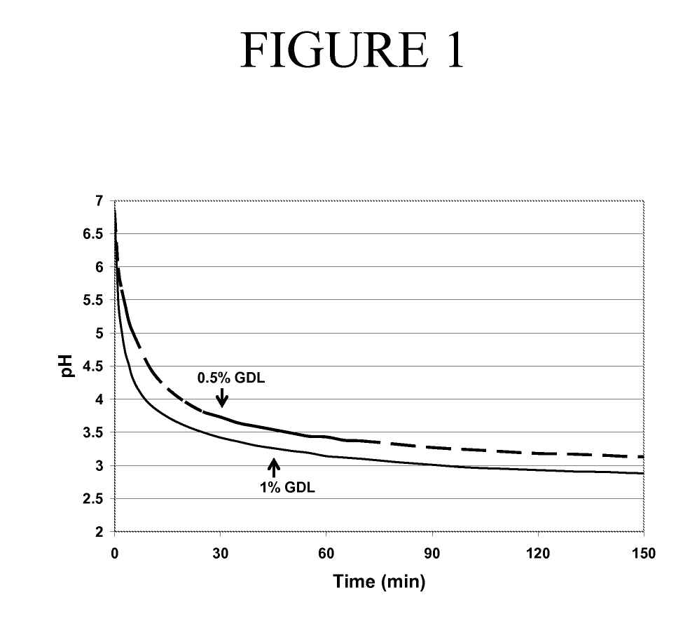

A composition of the invention comprises a gelation inducer, in particular, a gelation inducer comprising an acidifier. An acidifier, e.g., glucono-δ-lactone (GDL), can be used as a gelation inducer in a composition of the invention. GDL is preferred since it slowly releases acid which controls the gelation process. GDL is slowly hydrolyzed to be gluconic acid and gradually lowers pH over time (FIG. 1). When pH becomes low enough, a gel is formed. A higher GDL level resulted in faster gel set, while it negatively affected the sparkling clarity of gels. A rapid acidifier will gel the composition, but can negatively affect the homogeneity of the gel. For example, a strong acid will gel the composition immediately in the area of its addition and not disperse throughout the composition. It is desired that the acidifier lower the pH slowly enough to create a homogeneous gel. Acidifiers are known in the art and are commercially available. The amount of acidifier used in the gel composition is an amount effective to form an essentially homogeneous gel, e.g., about 0.5 to about 1.0 wt % of the total weight of the composition for GDL. One of ordinary skill in the art can choose an acidifier and determine an effective amount to homogeneously gel the composition.

A gel composition of the invention further comprises water. The amount of water is an amount effective to hydrate the gellan gum. The water can be part of an aqueous solution. An aqueous solution should not contain components which interfere with the structure of the gel (or additional components must be added which remove the negative effect of those components in the aqueous solution). One of ordinary skill in the art can choose appropriate aqueous solution(s) and determine an appropriate amount thereof to use in a composition of the invention.

Additional ingredients can be added to the gel of the composition. For example, in an air freshener gel, fragrance can be added, and optionally, color. One of ordinary skill in the art can choose additional components and determine appropriate amounts thereof based on a desired application.

Fragrance is known in the art and is commercially available or can be produced by known methods. The amount of fragrance can be readily determined by one of ordinary skill in the art. If a fragrance to be used is not water-soluble, it can be incorporated in a composition of the invention along with a surfactant or surfactant system, e.g., as a water-soluble emulsion.

A composition of the invention can be produced, for example, by a process described in the Methods and the Examples sections. A composition of the invention can be produced without any thermal treatment.

An air freshener gel can comprise a gel composition of the invention. An air freshener gel can comprise a deacylated gellan, an effective amount of a sequestrant, an effective amount of a syneresis control agent, a gelation inducer comprising an acidifier, and a fragrance.

### EXAMPLE EMBODIMENTS

A specific example gel of the invention can comprise 0.45 wt % deacylated gellan gum, 1.35 wt % syneresis control agent, 0.5 wt % acidifier, and 0.02 wt % sequestrant.

Another example gel of the invention can comprise 0.5 wt % deacylated gellan gum, 1.5 wt % syneresis control agent, 0.5 wt % acidifier, and 0.02 wt % sequestrant.

A third example gel of the invention can comprise 0.2 wt % deacylated gellan gum, 1.0 wt % syneresis control agent, 0.5 wt % acidifier, 0.03 wt % acid soluble calcium salt, and 0.02 wt % sequestrant.

### Methods

Described herein is the use of deacylated gellan (e.g., KELCOGEL® gellan, CP Kelco, Atlanta, Ga.) for isothermally preparing heat-resistant gels with little or no syneresis. A method of the invention comprises isothermal hydration and gelation of deacylated gellan. The isothermal process can be performed at room temperature (ambient conditions).

An air freshener gel (AFG) for delivering fragrance into the air and/or for neutralizing room odor is an application for a gel of the invention. The conventional production of gellan-based AFGs uses a heating process to dissolve and disorder the gellan gum. An alternative method is of great interest since a heating process causes evaporation of volatile fragrance which is the most expensive component in most AFGs.

Attempts were made at preparing an AFG without involving any thermal treatments by hydrating deacylated gellan (e.g., KELCOGEL® gellan) at room temperature using a sequestrant and then inducing the formation of an acid-set gel by incorporating glucono-δ-lactone (GDL) that slowly acidifies the system over time. However, resulting acid-set gels turned out to show massive syneresis.

It was found that combining deacylated gellan (e.g., KELCOGEL® gellan) and xyloglucan (e.g., tamarind seed xyloglucan (TSX)) reduced syneresis of acid-set gellan gels.

As further described in the Examples, example embodiments of acid-set gels were isothermally formed by utilizing a method comprising blending deacylated gellan gum, an effective amount of a sequestrant, an effective amount of a syneresis control agent (e.g., xyloglucan (TSX)), and a gelation inducer comprising an acidifier to form a blend; hydrating the blend at ambient conditions; and allowing the hydrated blend to rest at ambient conditions for a time effective to form a gel.

In the Examples, the ingredients were manually dry blended. The ingredients were blended for a time effective to form a homogeneous blend. Other methods of blending the ingredients (or equipment therefor) are known to one of ordinary skill in the art.

The ingredients can be added to a composition according to the invention individually rather than in a blend. If so, the acidifier should be added last so that a gel does not form prematurely. The sequestrant is added to help the gellan hydrate so it should preferably be added with or before the gellan gum.

The ingredients are described above in more detail in the COMPOSITION section.

Preferably, the weight ratio of xyloglucan to gellan gum is about 3:1. It is preferable that the gellan weight be less than one half percent of the total weight of the composition. The amount of xyloglucan (syneresis control agent) should be effective to prevent/control syneresis of the final composition, regardless of the gellan concentration.

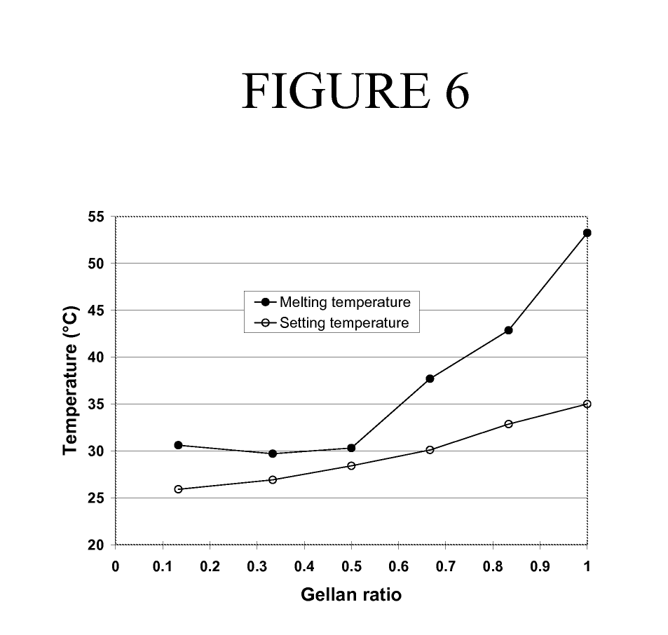

The gel melt temperature increased with increasing gellan weight ratio of the total gum weight, especially when there was a higher ion content in the gellan gum. The melt temperature of the final gel was almost constant when the gellan weight ratio was less than 0.5. At a gellan weight ratio >0.5, the melt temperature increased steeply with increasing gellan weight ratio. When the total gum level was fixed, the gel set temperature gradually increased with the gellan content (see, e.g., FIG. 6). This is most likely a reflection of higher ionic contents in the deacylated gellan gum (see, e.g., Table 4).

A higher GDL level resulted in faster gel set, while the higher acidifier level negatively affected the sparkling clarity of gels. Gels containing >0.4 wt % gellan gum were reasonably firm, i.e., a self standing gel.

Hydrating the blended ingredients is done by adding an amount of water (e.g., aqueous solution) effective to solubilize the components. The hydrated ingredients are preferably mixed for a time effective/sufficient to form a homogenous solution at essentially isothermal conditions (i.e., conditions under which fragrance will not degrade or be lost through evaporation). Methods of mixing hydrated ingredients are known to one of ordinary skill in the art. The ingredients are relatively resistant to shearing, therefore, mixing method, equipment, speed and the like are not believed to be critical.

Fragrance or other additives can be added, preferably after hydration of the gellan.

The hydrated solution is allowed to gel. This can be accomplished by letting the solution rest for a period of time effective to achieve gelation. This can be done at isothermal conditions, such as room temperature. Other ways can be used but this is preferred for cost effectiveness. Gel set time can be controlled by manipulating, for example, the level of acidifier.

The resulting gels had improved syneresis relative to gels without a TSX syneresis control agent. In example embodiments, syneresis (e.g., evaluated after two days storage at room temperature) was found to be negligible (based on visual observation) when the xyloglucan (e.g., TSX) content was more than three-fold that of the deacylated gellan gum (e.g., KELCOGEL® gellan) content by weight. These resulting gels were also confirmed to be fairly heat-stable; they did not melt at around 60° C.

The origin of the synergy between deacylated gellan and TSX was tentatively attributed to size-exclusion effects of xyloglucan.

### EXAMPLE EMBODIMENT

In a specific example embodiment of the present invention, KELCOGEL® gellan/TSX blend systems that can be both hydrated and gelled at room temperature were developed. A small amount of a sequestrant (sodium citrate) allowed KELCOGEL® gellan to be hydrated at room temperature without heating. Isothermal gelation was then realized using a glucono-δ-lactone acidifier that gradually acidified the system over time. The addition of TSX was found to be surprising in preventing unfavorable syneresis.

### Applications

The invention relates to food and non-food products comprising the gels of the invention.

### Food and Non-Food Products

The subject gels are useful as gels in, e.g., gelled pet foods, microbial and tissue culture media, liquid cleaners, toothpastes, soap and body washes, deodorant gels, air freshener gels, soft capsules, and other known applications of gels.

A heat resistant gel composition of the invention can be used for delivering fragrance, such as an air freshener gel preparation. Air freshener gel is used for delivering fragrance into the air and/or for neutralizing room odor. Use of a present composition and/or method will provide AFG manufacturers with an opportunity to reduce energy costs for production and to minimize loss of fragrance during production since the AFGs can be made at ambient temperature. An air freshener gel is preferably prepared without heating to avoid loss of fragrance. Additionally, final products should be fairly heat-stable so that they do not melt in a hot environment.

Initial trials with conventional gels resulted in massive syneresis especially in the presence of calcium. Thus, gels of the current invention can reduce syneresis by incorporating TSX and without the need for using calcium (though calcium can also be used as seen below).

### EXAMPLES

The following examples provide illustrations of the present invention and should not be misconstrued to limit in any way the scope of the present invention.

The following examples are put forth so as to provide those of ordinary skill in the art with a complete disclosure and description of how the compositions, articles, and/or methods described and claimed herein are made and evaluated, and are intended to be purely exemplary, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product obtained from the described process. Only reasonable and routine experimentation will be required to optimize such process conditions.

### Example 1

### Proof of Concept

The concept of preparing acid-set gellan gels with reduced syneresis by combining xyloglucan, i.e., TSX, with deacylated gellan gum was tested.

Acid-set gels were isothermally formed from dry-blended powders containing sodium citrate (0.02 wt %), deacylated gellan gum (KELCOGEL® gellan) (0.2-0.75 wt %), xyloglucan (TSX) (0-1.5 wt %), and acidifier (GDL) (0.5-1 wt %). Powders of sodium citrate, KELCOGEL® gellan (CP Kelco, Atlanta, Ga.), TSX (Glyloid 6C, Dainippon Sumitomo Pharma, Osaka, Japan), and GDL were dry-blended and dissolved into deionized (DI) water with vigorous stirring for 5-10 minutes at room temperature. Then, at rest, gels appeared to set in 30-60 minutes in the presence of 0.5 wt % GDL at ambient conditions.

Faster set was observed at 1.0 wt % GDL, while the gels became slightly cloudy. A higher GDL level resulted in faster gel set, but the higher GDL level negatively affected the sparkling clarity of gels.

In a separate experiment, aqueous solutions of 0.5 wt % and 1.0 wt % GDL showed pH values of 2.5 and 2.3, respectively, after two days of equilibration at room temperature. Acidification profiles of GDL solutions were measured using a standard pH meter (Beckman). As shown in FIG. 1, it takes about 25 and 50 min for 1% and 0.5% GDL solutions containing 0.02% sodium citrate and 1.5% TSX to reach pH 3.5, respectively. GDL hydrolysis produced a reproducible pH reduction which provides a way to control the gelation rate of the gellan.

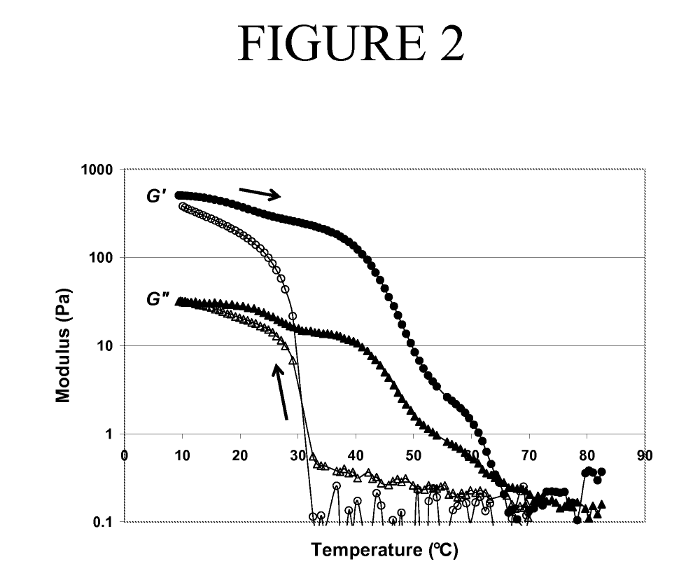

An acid-set gel of deacylated gellan is supposed to exhibit thermal hysteresis. Published atomic force microscopy images of acid-set gellan gels (Gunning, et al., “Corrections: Investigation of gellan networks and gels by atomic force microscopy,” Macromolecules, 30, 163-164, 1997) reveal that the gel network is composed of thick fibrous strands that are considered to represent bundles of laterally associated double helical gellan molecules. FIG. 2 confirms that an acid-set gel shows relatively wide thermal hysteresis.

A KELCOGEL® gellan concentration over 0.4 wt % produced a self-standing gel. These gels were confirmed to be reasonably heat-stable; the gels did not melt by heating at 60° C. Gel-set/melt profiles of a GDL-induced gel were determined using a controlled-stress Bohlin rheometer equipped with a cone and plate test fixture. Difficulties in handling due to high viscosity during gel preparation were experienced at KELCOGEL® gellan concentrations over 0.5 wt %.

Results of the degree of syneresis evaluated after more than 2 days of storage at room temperature are summarized in Table 1. Syneresis was found to be negligible when the TSX content was 3 times or more by weight than the gellan content. Based on these results, systems with 0.45% KELCOGEL® gellan/1.35% TSX/0.5% GDL and 0.5% KELCOGEL® gellan/1.5% TSX/0.5% GDL were selected as of most interest from those tested.

TABLE 1
Effects of TSX on syneresis of acid-set gellan gels prepared without
thermal treatment. Syneresis represents the weight (g) of water released
from a 100 g gel after >2 days of storage at room temperature.

| | KELCOGEL ® | | | |
|---|---|---|---|---|
| Na citrate | gellan | TSX | GDL | Syneresis |
| (%) | (%) | (%) | (%) | (g) |
| 0.02 | 0.2 | 1 | 0.5 | 0 |
| 0.02 | 0.2 | 1 | 1.0 | 0 |
| 0.02 | 0.3 | 1 | 0.5 | 0 |
| 0.02 | 0.3 | 1 | 1.0 | 0 |
| 0.02 | 0.4 | 1.2 | 0.5 | 0 |
| 0.02 | 0.4 | 1.2 | 1.0 | 0 |
| 0.02 | 0.45 | 1.35 | 0.5 | 0 |
| 0.02 | 0.45 | 1.35 | 1.0 | 0 |
| 0.02 | 0.5 | 0 | 0.5 | 7.0 |
| 0.02 | 0.5 | 0 | 1.0 | 7.5 |
| 0.02 | 0.5 | 0.5 | 0.5 | 3.5 |
| 0.02 | 0.5 | 0.5 | 1.0 | 4.1 |
| 0.02 | 0.5 | 1 | 0.5 | 0.9 |
| 0.02 | 0.5 | 1 | 1.0 | 0 |
| 0.02 | 0.5 | 1.5 | 0.5 | 0 |
| 0.02 | 0.5 | 1.5 | 1.0 | 1.0 |

### Example 2

### Use of Other Gelation Inducers

### Calcium Lactate

More rapid gel-set was intended by trying calcium lactate in place of GDL. Powders of sodium citrate, KELCOGEL® gellan, and TSX were dry-blended and dissolved into DI water with vigorous stirring for 5-10 min at room temperature. Stirring was stopped immediately after powders of calcium lactate were dispersed into the hydrated blend because the resulting very high viscosity made mixing extremely difficult. Because of the high viscosity of the sample sol, calcium powders remained suspended without stirring and visible for about 20 min. The blend turned into a gel in several minutes at rest. The TSX in the composition created homogeneity of the final gel structure and also prevented syneresis.

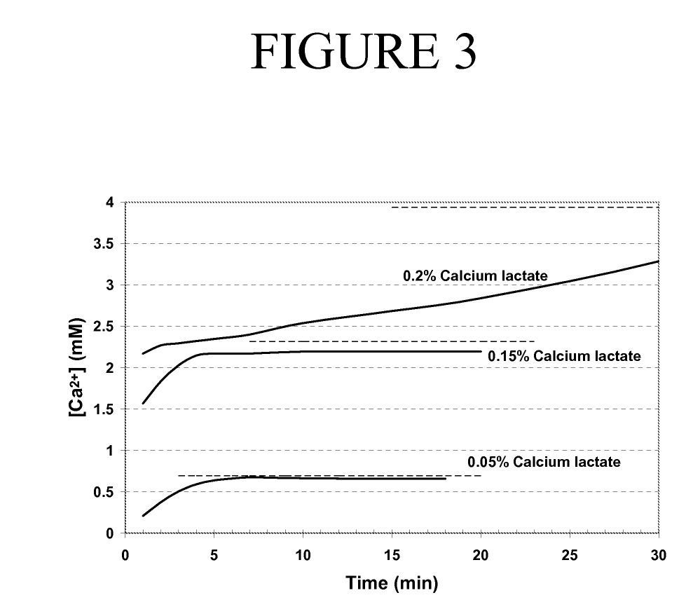

The calcium ion concentration was shown to increase rather rapidly (FIG. 3), consistent with the observation of rapid gel-set after preparation.

When utilizing GDL as the gelation inducer (Example 1), a KELCOGEL® gellan concentration over 0.4 wt % was required to obtain a self-standing gel. The present example allowed a gum level as low as 0.2 wt % to result in a reasonably strong gel as shown in Table 2.

TABLE 2
Effects of various compositions on syneresis of calcium lactate set gellan
gels prepared without thermal treatment.

| | KELCOGEL ® | | | |
|---|---|---|---|---|
| Na citrate | gellan | TSX | Ca lactate | Syneresis |
| (%) | (%) | (%) | (%) | (%) |
| 0.05 | 0.2 | 1.0 | 0.10 | 0 |
| 0.05 | 0.2 | 1.0 | 0.15 | 0 |
| 0.05 | 0.3 | 1.0 | 0.10 | 0 |
| 0.05 | 0.3 | 1.0 | 0.15 | 0 |
| 0.05 | 0.4 | 1.2 | 0.10 | 0 |
| 0.05 | 0.4 | 1.2 | 0.15 | 0 |
| 0.05 | 0.5 | 1.5 | 0.10 | 0 |
| 0.05 | 0.5 | 1.5 | 0.15 | 0 |

Additionally, the clarity of these gels appeared to be better than acid-induced gels; however, a disadvantage is the almost instantaneous gel-set which can limit flexibility in process design.

### Acid-Soluble Calcium

Calcium phosphate is practically insoluble in ambient water, but it becomes soluble in acidic conditions. Based on this solubility difference, controlled release of calcium ions from calcium phosphate was attempted by controlling pH using GDL.

All powdered ingredients were dry-blended together and dissolved into DI water with vigorous stirring for 5 min at room temperature. Resulting sols appeared to be cloudy due to the presence of insoluble calcium. Gel-set occurred around 20-30 min after preparation. Clear gels with reasonable mechanical strengths were obtained overnight. TSX played a significant role in reducing syneresis (Table 3).

TABLE 3
Effects of various compositions on syneresis of acid soluble
calcium/acid set gellan gels prepared without thermal treatment.

| | KELCOGEL ® | | | | |
|---|---|---|---|---|---|
| Na citrate | gellan | TSX | GDL | CaHPO₄ | Syneresis |
| (%) | (%) | (%) | (%) | (%) | (%) |
| 0.02 | 0.2 | 0 | 0.5 | 0.03 | 21 |
| 0.02 | 0.2 | 0 | 0.5 | 0.05 | 21 |
| 0.02 | 0.5 | 0 | 0.5 | 0.03 | 24 |
| 0.02 | 0.5 | 0 | 0.5 | 0.05 | 24 |
| 0.02 | 0.2 | 1.0 | 0.5 | 0.03 | 0 |
| 0.02 | 0.2 | 1.0 | 0.5 | 0.05 | 0.6 |
| 0.02 | 0.3 | 1.2 | 0.5 | 0.03 | 0.6 |
| 0.02 | 0.4 | 1.2 | 0.5 | 0.03 | 4.6 |
| 0.02 | 0.5 | 1.5 | 0.5 | 0.03 | 3.8 |
| 0.02 | 0.5 | 1.5 | 0.5 | 0.05 | 4.3 |

These gels were confirmed to maintain their structural integrity when heated at 85° C. for 30 min.

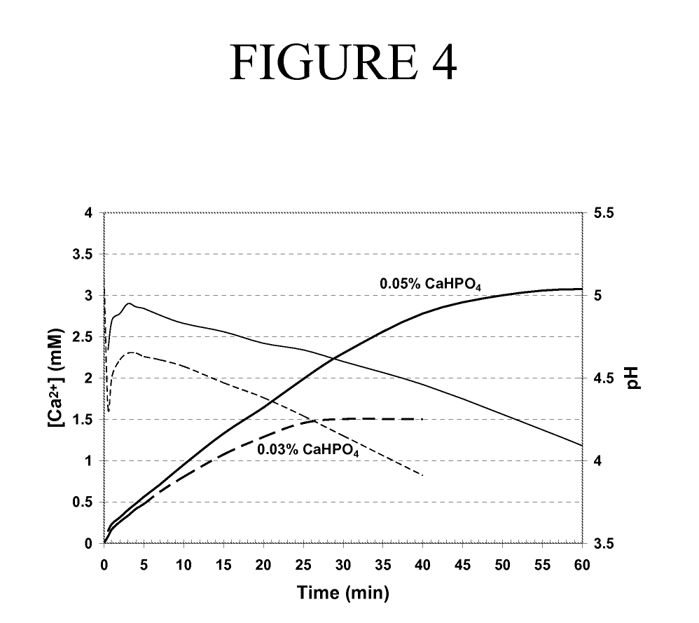

The rate of increase in calcium ion concentration with the calcium lactate use was shown to be controllable using an acid-soluble calcium salt together with GDL. FIG. 4 shows a time-dependent dissolution of calcium phosphate and a concurrent decrease in pH. The calcium concentration remained below 1 mM for the first 10 min, continued to increase gradually with time, and reached an equilibrium value in >25 min. Such slow increases in calcium concentration secured sufficient time for gel preparation.

### Example 3

### Mixing Ratio Gellan Gum and TSX

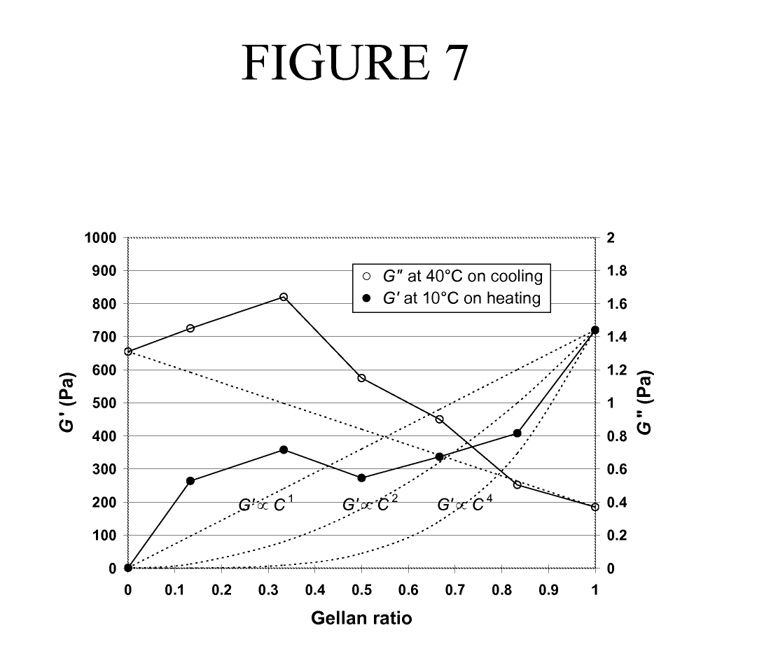

Effects of the mixing ratio of deacylated gellan gum (KELCOGEL® gellan) and tamarind seed xyloglucan (TSX) on their interactions were rheologically evaluated at the total gum level of 1.5 wt %. Synergistic effects on the storage modulus at 10° C. were remarkable when the gellan ratio was less than 0.5. FIG. 7 shows that storage modulus values at 10° C. are higher than mean values if the gellan ratio is between about 0.1 and about 0.4. The gel melt temperature increased with increasing gellan ratio, reflecting higher ion contents in KELCOGEL® gellan.

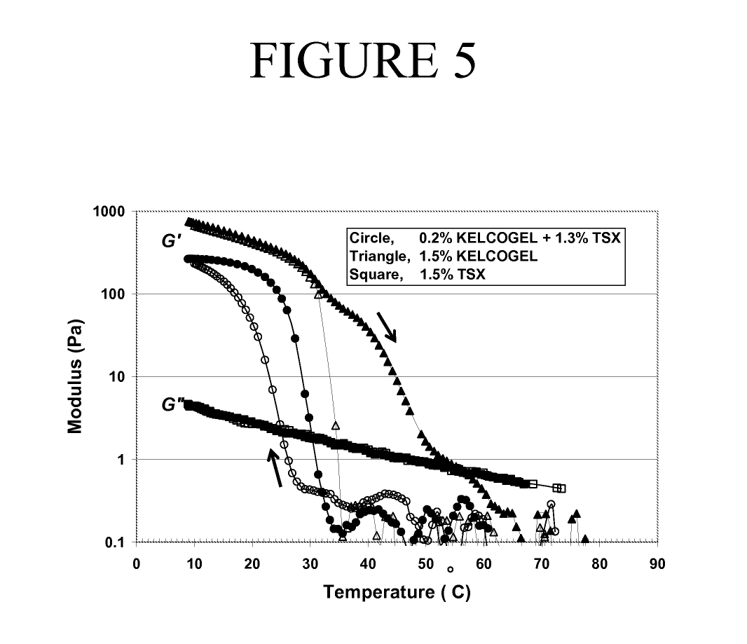

FIG. 5 confirmed that a 1.5 wt % aqueous solution of TSX (Glyloid 6C, Dainippon Sumitomo Pharma, Osaka, Japan) did not show any transitional changes in the temperature dependence of the loss modulus in the temperature range between 10° C. and 70° C. When a small portion (0.2%) of TSX was replaced with KELCOGEL® gellan, the system formed a gel on cooling. The storage modulus value reached >260 Pa at 10° C., while the gel melted around 30° C. on heating. This KELCOGEL® gellan does not form a gel at the use level of 0.2% if no additional salts are added. A stronger gel was formed from 1.5% KELCOGEL® gellan alone (FIG. 5), but the gel showed a much higher melt temperature over 50° C.

When the total gum level was fixed to 1.5 wt %, the gel set temperature gradually increased with the gellan content (FIG. 6). This is most likely to reflect higher ionic contents in KELCOGEL® gellan (Table 4). The melt temperature was almost constant when the gellan weight ratio was less than 0.5. At the gellan weight ratio >0.5, the melt temperature increased steeply with increasing gellan weight ratio.

TABLE 4
Elemental analysis results for major cationic components of the gums.

| | Ca | Na | Mg | K |
|---|---|---|---|---|
| | (ppt) | (ppt) | (ppt) | (ppt) |
| KELCOGEL ® | 2.633 | 4.762 | 0.847 | 49.340 |
| gellan | | | | |
| TSX | 0.202 | 0.173 | 0.139 | 0.123 |

FIG. 7 demonstrates synergistic interactions between KELCOGEL® gellan and TSX. Storage modulus values determined at 10° C. were larger than the arithmetic means of values for individual systems when the gellan weight ratio was less than 0.5. At a higher gellan weight ratio, storage modulus values were lower than the arithmetic means, but still larger than values expected based on a hypothetical power law relationship between the storage modulus and gellan concentration (G′∝C⁴). The cubic relationship between the gellan weight ratio and storage modulus indicated that synergistic effects of xyloglucan becomes suppressed by increasing ionic concentration at higher gellan wt ratios.

Synergy between deacylated gellan gum and TSX was also observed at a temperature above the sol-gel transition temperature. In FIG. 7, loss modulus values determined at 40° C. on initial cooling were plotted against the gellan weight ratio. As previously demonstrated, most values are above the arithmetic means of values for individual systems, demonstrating synergistic interactions once again. Since this temperature is above the sol-gel transition temperature, gellan molecules are supposed to be in the disordered state. In other words, the thermal energy is sufficiently high to break hydrogen bonds that stabilize double-stranded helical structures of gellan. Therefore, it does not seem quite rational to assume the presence of intermolecular binding between gellan and xyloglucan at such a high temperature. Furthermore, it has been reported in the literature (Nitta, et al., “Synergistic gel formation of xyloglucan/gellan mixtures as studied by rheology, DSC, and circular dichroism,” Biomacromolecules, 4, 1659, 2003) that the ellipticity that reflects molecular environment around the carboxyl group of gellan is not influenced by the presence of xyloglucan at temperatures above the transition temperature. Since the ellipticity is known to be highly sensitive to conformational transitions of gellan molecules, the lack of influence of xyloglucan on the ellipticity seems to suggest the absence of intermolecular binding between gellan and xyloglucan.

### Example 4

### Prophetic Example

### Air Freshener Gel

An application of the present gel is an air freshener gel. An air freshener gel is preferably prepared without heating to avoid loss of fragrance. Additionally, final products should be fairly heat-stable so that they do not melt in a hot environment.

Three compositions are tested as AFGs based on previous testing: 0.45% KELCOGEL® gellan/1.35% TSX/0.5% GDL/0.02% sodium citrate, 0.5% KELCOGEL® gellan/1.5% TSX/0.5% GDL/0.02% sodium citrate, and 0.2% KELCOGEL® gellan/1.0% TSX/0.5% GDL/0.03% CaHPO₄/0.02% sodium citrate. An effective amount of fragrance and, optionally, surfactant are added to these 3 test compositions.

Once these model AFG systems are prepared, the systems are evaluated for gel set time, gel strength, and syneresis.

While the present invention is described above with respect to what is currently considered to be its preferred embodiments, it is to be understood that the invention is not limited to that described above. To the contrary, the invention is intended to cover various modifications and equivalent arrangements included within the spirit and scope of the appended claims.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed is:

1. A gel comprising
a deacylated gellan gum,
an effective amount of a sequestrant,
an effective amount of a syneresis control agent, and
a gelation inducer.

2. The gel of claim 1, wherein the gelation inducer comprises an acidifier.

3. The gel of claim 1, wherein the sequestrant is sodium citrate, EDTA, sodium hexametaphosphate, or mixtures thereof.

4. The gel of claim 1, wherein the sequestrant is sodium citrate.

5. The gel of claim 1, wherein the syneresis control agent is xyloglucan.

6. The gel of claim 5, wherein the xyloglucan is tamarind seed xyloglucan (TSX).

7. The gel of claim 2, wherein the acidifier is glucono-δ-lactone (GDL).

8. The gel of claim 1, wherein the gelation inducer comprises calcium lactate.

9. The gel of claim 1, wherein the gelation inducer comprises GDL and an acid soluble calcium salt.

10. The gel of claim 1, wherein the deacylated gellan gum is about 0.2 to about 0.5 wt % of the gel.

11. The gel of claim 6, wherein the TSX is about 1 to about 1.5 wt % of the gel.

12. The gel of claim 7, wherein the GDL is about 0.5 wt % of the gel.

13. The gel of claim 4, wherein the sodium citrate is about 0.02 wt % of the gel.

14. The gel of claim 1, wherein the weight ratio of syneresis control agent to deacylated gellan gum is about 3:1.

15. The gel of claim 1, wherein the amount of syneresis control agent is effective to keep an amount of syneresis of the gel below about 5%.

16. The gel of claim 1 further comprising fragrance.

17. The gel of claim 1, wherein the gel comprises 0.45% deacylated gellan gum, 1.35% syneresis control agent, 0.5% acidifier, and 0.02% sequestrant.

18. The gel of claim 1, wherein the gel comprises 0.5% deacylated gellan gum, 1.5% syneresis control agent, 0.5% acidifier, and 0.02% sequestrant.

19. The gel of claim 1, wherein the gel comprises 0.2% deacylated gellan gum, 1.0% syneresis control agent, 0.5% acidifier, 0.03% acid soluble calcium salt, and 0.02% sequestrant.

20. A method of preparing a heat-resistant gel comprising
(a) blending a deacylated gellan gum, an effective amount of a sequestrant, an effective amount of a syneresis control agent, and a gelation inducer comprising an acidifier to form a blend;
(b) hydrating the blend with an amount of water; and
(c) resting the hydrated blend until a gel forms,
wherein the blending, hydrating and resting are performed essentially isothermally at ambient conditions.

21. The method of claim 20, wherein the syneresis control agent is tamarind seed xyloglucan (TSX).

22. The method of claim 20, wherein the acidifier is glucono-δ-lactone (GDL).

23. A gel prepared by the method of claim 20.

24. An air freshener product comprising the gel of claim 16.

25. A process for preparing a gel comprising the steps of:
isothermally hydrating deacylated gellan at ambient temperature;
mixing the hydrated deacylated gellan gum and a tamarind seed xyloglucan;
gelling the mix by addition of gelation inducer.

26. The process of claim 25 wherein the gelation inducer comprises an acidifier.

27. The process of claim 25 wherein the isothermal hydration comprises adding an effective amount of a sequestrant and water to the deacylated gellan.
